(19) United States
(12) Patent Application Publication
(10) Pub. No.: US 2007/0073119 A1
(43) Pub. Date: Mar. 29, 2007

(54) Wireless network connected pulse oximeter

(76) Inventors: James Wobermin, Arvada, CO (US); Christopher Crowley, Golden, CO (US); James Galbiati, Boulder, CO (US); Jeffrey Gentry, Castle Rock, CO (US)

Correspondence Address: MARSH, FISCHMANN & BREYFOGLE LLP, 3151 SOUTH VAUGHN WAY, SUITE 411, AURORA, CO, 80014, US

(21) Appl. No.: 11/238,648

(22) Filed: Sep. 29, 2005

(51) Int. Cl. A61B 5/00

(52) U.S. Cl. 600/323

(57) ABSTRACT

A wireless network connected pulse oximetry system and method of providing pulse oximetry data obtained by a pulse oximeter to a monitoring station that is remote from a patient monitored by the pulse oximeter are provided. In one embodiment, a wireless network connected pulse oximetry system (100) includes a pulse oximeter (110) having an optical data transmitter (112) such as an infra-red LED and associated LED drive circuitry). The system (110) also includes a computer (140) that is interconnectable with a global data network (104) (e.g., the Internet) and an optical data receiver (130) that is connectable with a data port (144) of the computer via a data cable (142). The optical data receiver (130) is operable to receive optically transmitted pulse oximetry data (102) from the pulse oximeter (110) and convert the received pulse oximetry data (102) for transmission via the data cable (142) to the data port (144) of the computer (140). The system also includes a software module (146) executable by the computer (140) that enables the computer (140) to format the pulse oximetry data (102) received on its data port (144) for transmission via the global data network (104) to a remote monitoring station (150). In other embodiments, the optical data receiver may not be necessary, or the pulse oximetry data may instead be transmitted via a radio-frequency wireless connection.

### FIELD OF THE INVENTION

The present invention relates generally to patient medical monitoring devices, and more particularly to enabling pulse oximeters for wireless Internet connectivity.

### BACKGROUND OF THE INVENTION

Photoplethysmographic systems such as pulse oximeters utilize light signals corresponding with two or more different center wavelengths to non-invasively determine various blood analyte concentrations in a patient's blood and to obtain information regarding the patient's heart rate and the like. By way of primary example, blood oxygen saturation (SpO₂) levels of a patient's arterial blood are monitored in pulse oximeters by measuring the absorption of oxyhemoglobin (O2Hb) and reduced hemoglobin (RHb) using red and infrared light signals. The measured absorption data allows for the calculation of the relative concentrations of O2Hb and RHb, and therefore Sp0₂ levels, since RHb absorbs more light than O2Hb in the red band and O2Hb absorbs more light than RHb in the infrared band, and since the absorption relationship of the two analytes in the red and infrared bands is known.

To obtain absorption data, pulse oximeters typically comprise a probe that is releaseably attached to a patient tissue site (e.g., finger, ear lobe, nasal septum, foot). The probe directs red and infrared light signals through the patient tissue site. The light signals are provided by one or more light signal sources (e.g., light emitting diodes or laser diodes) which are typically disposed in the probe. A portion of the red and infrared light signals is absorbed in the patient tissue site and the intensity of the transmitted light signals (light exiting the patient tissue site is referred to as transmitted) is detected by a detector that may also be located in the probe. The detector outputs a signal which includes information indicative of the intensities of the transmitted red and infrared light signals. The output signal from the detector may be processed to obtain separate signals associated with the red and infrared transmitted light signals (i.e., separate red and infrared plethysmographic signals or waveforms).

It is sometimes desirable to have a pulse oximeter that is a relatively small, handheld, and easily portable device. Such small handheld portable pulse oximeters are useful for emergency medical personnel and the like since they can be easily transported to an emergency site and used to monitor a patient who is being transported without the inconvenience of relatively bulky equipment. Such handheld pulse oximeters are also useful for hospital situations where patients are being transported from between rooms. However, due to their limited size, such handheld pulse oximeters may sometimes have limited pulse oximetry data analysis and display capabilities. Thus it is sometimes desirable to connect such handheld pulse oximeters to other devices for additional data analysis and display capabilities.

### SUMMARY OF THE INVENTION

Accordingly, the present invention provides a wireless network connected pulse oximetry system and method of providing pulse oximetry data obtained by a pulse oximeter to a monitoring station that is remote from a patient monitored by the pulse oximeter. The pulse oximetry system and method of the present invention provide for the wireless connection of a pulse oximeter or the like to a data network such as, for example, the Internet. Pulse oximetry data obtained by the pulse oximeter from a patient may then be accessed by a remote monitoring station connected to the data network. The remote monitoring station may provide enhanced display and/or data analysis capabilities and thus, the pulse oximetry system and method of the present invention are particularly advantageous in the context of relatively small handheld portable pulse oximeters.

According to one aspect of the present invention, a wireless network connected pulse oximetry system includes a pulse oximeter including an optical data transmitter. The pulse oximeter is operable to obtain pulse oximetry data from a patient, and the optical data transmitter is operable to optically transmit the pulse oximetry data obtained from the patient. In this regard, the optical data transmitter may, for example, be comprised of an infra-red LED and associated LED drive circuitry that is operable to modulate the intensity of the LED (e.g., between on condition and an off condition). The system also includes a computer that is interconnectable with a global data network (e.g., the Internet) and an optical data receiver that is connectable with a data port of the computer via a data cable. The optical data receiver is operable to receive optically transmitted pulse oximetry data from the pulse oximeter and convert the received pulse oximetry data for transmission via the data cable to the data port of the computer. In this regard, the optical data receiver may, for example, include a photodetector that is sensitive to infra-red wavelength optical signals, and may, for example, be operable to convert the received optical signals to a serial data stream for transmission via a serial data cable to a serial port of the computer. The system also includes a software module executable by the computer that enables the computer to format the pulse oximetry data received on its data port for transmission via the global data network to a remote monitoring station.

The system may also include one or more data storage devices. For example, a data storage device (e.g., a memory chip) may be included in the pulse oximeter for storing the pulse oximetry data after the pulse oximetry data is obtained from the patient This allows the pulse oximeter to be used to collect data without requiring it to be located in an appropriate relationship with respect to the optical data receiver for immediate optical transmission of the data therebetween. Instead, the data stored in the data storage device of the pulse oximeter may be transmitted at a later time to the optical data receiver. By way of further example, the computer may include a data storage device (e.g., a hard drive, a floppy drive, an optical media drive, or a tape drive) for storing the pulse oximetry data after the pulse oximetry data is received on its data port. This allows the data to be received by the computer and stored for some period of time until the computer can be connected to the global data network.

In accordance with another aspect of the present invention, the wireless network connected pulse oximetry system does not include an optical data receiver. Rather, the computer includes an optical data port (e.g., a photodetector sensitive to infra-red wavelength optical signals) that is operable to receive optically transmitted data. In this regard, the software module enables the computer to format the pulse oximetry data received on the optical data port for transmission via the global data network to a remote monitoring station.

According to a further aspect of the present invention, a method of providing pulse oximetry data obtained by a pulse oximeter to a monitoring station that is remote from a patient monitored by the pulse oximeter includes the step of connecting an optical data receiver by a data cable to a data port of a computer that may be interconnected with a global data network. In this regard, the optical data receiver may, for example, be connected by a serial data cable to a serial port of the computer, and the global data network may, for example, comprise the Internet. The pulse oximeter and the optical data receiver are positioned relative to each other for optical transmission of the pulse oximetry data therebetween. In this regard, positioning the pulse oximeter and the optical data receiver may involve aligning an LED of the pulse oximeter in a line of sight relationship with a photodetector of the optical data receiver. The pulse oximetry data is optically transmitted from the pulse oximeter and is received by the optical data receiver. The received optically transmitted pulse oximetry data is converted to a format (e.g., serial data) appropriate for transmission via the data cable to the data port of the computer. The converted pulse oximetry data is transmitted from the optical data receiver and received on the data port of the computer. The pulse oximetry data received on the data port is formatted for transmission over the global data network, and then transmitted over the global data network to the remote monitoring station.

The pulse oximetry data that is provided to the remote monitoring station may be data that has been previously obtained and stored. In this regard, the method may further include the steps of operating the pulse oximeter to obtain the pulse oximetry data and storing the pulse oximetry data obtained in the operating step on a data storage device (e.g., a memory chip) of the pulse oximeter. This allows the pulse oximeter and optical data receiver to be mutually positioned in an appropriate relationship after the patient is monitored. For example, the pulse oximeter can be used to monitor the patient in one location (e.g., at an accident scene or in an ambulance) and the pulse oximetry data can be downloaded therefrom to the optical data receiver in another location (e.g., at a hospital). Alternatively, the pulse oximeter may be operated to obtain the pulse oximetry data while simultaneously optically transmitting the pulse oximetry data to the optical data receiver (with some lag time between obtaining the data and its optical transmission due to processing of the obtained data for optical transmission).

The converted pulse oximetry data may be simultaneously received on the data port of the computer, formatted for transmission over the global data network, and transmitted over the global data network to the remote monitoring station (with some possible lag time between reception of the data on the data port of the computer and transmission of the formatted data over the global data network due to the formatting process). Alternatively, the pulse oximetry data received on the data port of the computer may be stored on a data storage device of computer before it is transmitted over the global data network. In this regard, the pulse oximetry data may be formatted for transmission over the global data network before it is stored on the data storage device of the computer, or it may be formatted after being stored and prior to transmission over the global network upon request for the data by a remote monitoring station.

According to a further aspect of the present invention, the steps involving the optical data receiver need not be included. Rather, the method of providing pulse oximetry data obtained by a pulse oximeter having an optical data transmitter to a monitoring station that is remote from a patient monitored by the pulse oximeter includes the step of positioning the pulse oximeter and a computer having an optical data port and interconnectable with a global data network for optical transmission of the pulse oximetry data therebetween. In this regard, positioning the pulse oximeter and the computer may involve aligning an LED of the pulse oximeter in a line of sight relationship with a photodetector of the optical data port of the computer. The pulse oximetry data is optically transmitted from the pulse oximeter and is received by the optical data port of the computer. The pulse oximetry data received on the optical data port may then be formatted for transmission over the global data network and transmitted over the global data network to the remote monitoring station.

According to one more aspect of the present invention, a wireless network connected pulse oximetry system includes a pulse oximeter including a radio frequency (RF) data transmitter. The pulse oximeter is operable to obtain pulse oximetry data from a patient. The RF data transmitter is operable to broadcast an RF signal modulated to include the pulse oximetry data. The system also includes an RF data receiver interconnectable with a global data network (e.g. the Internet). In this regard, the RF data transmitter and the RF receiver may comprise wireless fidelity (WiFi) type devices. The RF data receiver is operable to receive the RF signal broadcast by the pulse oximeter and to convert the pulse oximetry data obtained from the received RF signal for transmission via the global data network to one or more remote monitoring stations.

In certain instances, the RF transmitter of the pulse oximeter and the RF receiver may not always be within suitable range of one another. In this regard, the system may include a data storage device (e.g., a memory chip) for storing the pulse oximetry data in the pulse oximeter after the pulse oximetry data is obtained from the patient. Once the RF transmitter of the pulse oximeter and the RF receiver are within suitable range of one another, the stored pulse oximetry data may then be transmitted.

These and other aspects and advantages of the present invention will be apparent upon review of the following Detailed Description when taken in conjunction with the accompanying figures.

### DESCRIPTION OF THE DRAWINGS

For a more complete understanding of the present invention and further advantages thereof, reference is now made to the following Detailed Description, taken in conjunction with the drawings, in which:

FIG. 1 is a block diagram illustrating one embodiment of a wireless Internet connected pulse oximetry system in accordance with the present invention;

FIG. 2 is a flowchart illustrating one manner of using the wireless Internet connected pulse oximetry system of FIG. 1 to provide pulse oximetry data to a remote monitoring station in accordance with the present invention;

FIG. 3 is a block diagram illustrating another embodiment of a wireless Internet connected pulse oximetry system in accordance with the present invention;

FIG. 4 is a flowchart illustrating one manner of using the wireless Internet connected pulse oximetry system of FIG. 3 to provide pulse oximetry data to a remote monitoring station in accordance with the present invention; and

FIG. 5 is a block diagram illustrating a further embodiment of a wireless Internet connected pulse oximetry system in accordance with the present invention.

### DETAILED DESCRIPTION

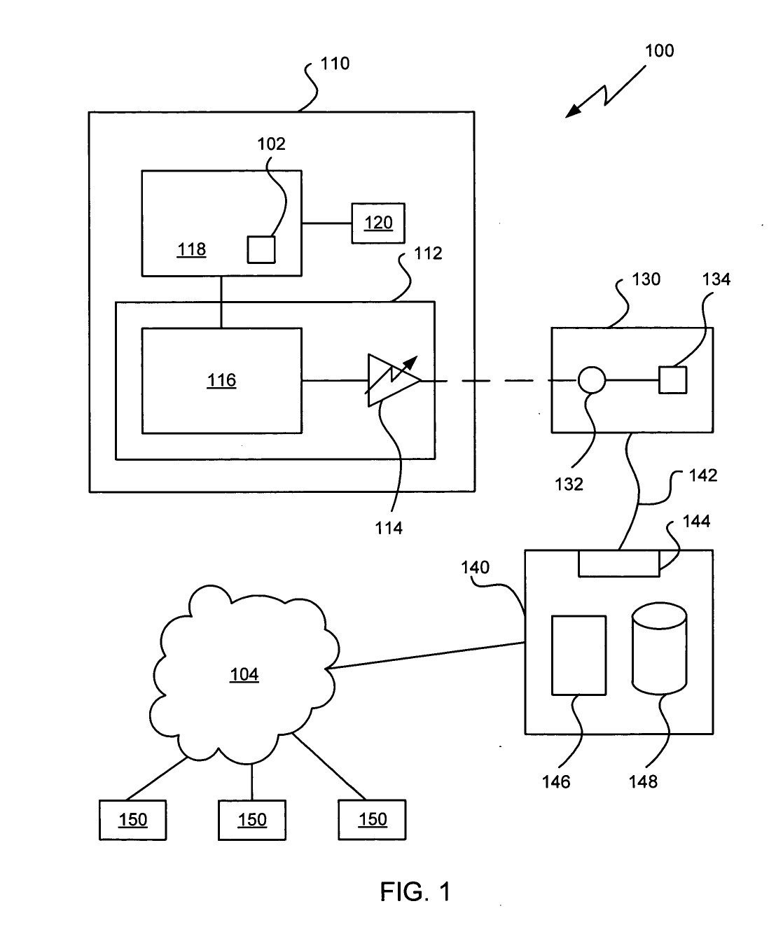

Referring now to FIG. 1 there is shown a block diagram of one embodiment of a wireless Internet connected pulse oximetry system 100. System 100 generally includes a pulse oximeter 110, an optical data receiver 130, and a computer 140 (e.g., a desktop, laptop or handheld computer or the like). The pulse oximeter 110 includes an optical data transmitter 112. Optical data transmitter 112 may, for example, comprise an infrared light emitting diode (LED) 114 and related LED drive circuitry 116. The LED drive circuitry 116 is operable to receive oximetry data 102 (e.g., a digitized plethysmographic waveform) from a processor 118 of the pulse oximeter 110 and modulate the LED 114 to optically transmit the oximetry data 102. In this regard, the pulse oximeter 110 may comprise a relatively small, portable pulse oximeter unit having a built in LED 114 and LED drive circuitry 116 such as the Datex-Ohmeda TUFFSAT® handheld pulse oximeter.

The optical data receiver 130 is connected via a data cable 142 to a data port 144 of the personal computer 140. In the presently described embodiment, data port 144 is a serial port and data cable 142 is a serial cable. However, data port 144 might instead be a parallel port, a universal serial bus, an IEEE 1394 port, or any other type of port enabling the personal computer 140 for receiving data from another device, with data cable 142 also being appropriately configured. The optical data receiver 130 includes a photodetector 132 or the like for receiving the optically transmitted pulse oximetry data 102 from the LED 114 of the pulse oximeter 110. In this regard, LED 114 and photodetector 132 should generally be maintained in a line of sight relationship with each other and within a suitable range of one another in order for the optically transmitted pulse oximetry data 102 to be received. Thus, it is desirable that the pulse oximeter 110 also include temporary data storage 120 (e.g., random access memory, flash memory) for storing the pulse oximetry data 102 for some period of time until the LED 114 and photodetector 132 can be brought into a suitable relationship with one another at which time the stored pulse oximetry data 102 may be transmitted. The optical data receiver 130 also includes processing hardware 134 (e.g., an appropriately programmed general purpose digital processor or an application specific integrated circuit) that converts the optically transmitted pulse oximetry data 102 received by the photodetector 132 into appropriately formatted serial data for transmission through the data cable 142 to the data port 144 of the computer 140.

The computer 140 is connected to the Internet 104 via, for example, a modem connected to an Internet Service Provider (ISP) server or a server of a local area network connected to the Internet 104. The computer 140 includes an appropriately configured software module 146 that, when executed by the computer 140, takes the pulse oximetry data 102 received from the data cable 142 on the data port 144 and formats the pulse oximetry data 102 for transmission over the computer's Internet connection to other devices (e.g., remote pulse oximetry monitoring and/or processing devices) connected to the Internet 104. Thus, pulse oximetry data 102 obtained by the pulse oximeter 110 is made available via the Internet 104 to monitoring stations 150 (e.g., other computers) that are geographically remote from the location of the patient being monitored by the pulse oximeter 110. In this regard, the computer 140 may include a data storage device 148 (e.g., a hard drive or a CDRW drive) for storing the pulse oximetry data 102 for some period of time until the pulse oximetry data 102 is requested by a remote monitoring station 150, at which time the pulse oximetry data 102 is transmitted by the computer via the Internet 104 to the remote monitoring station.

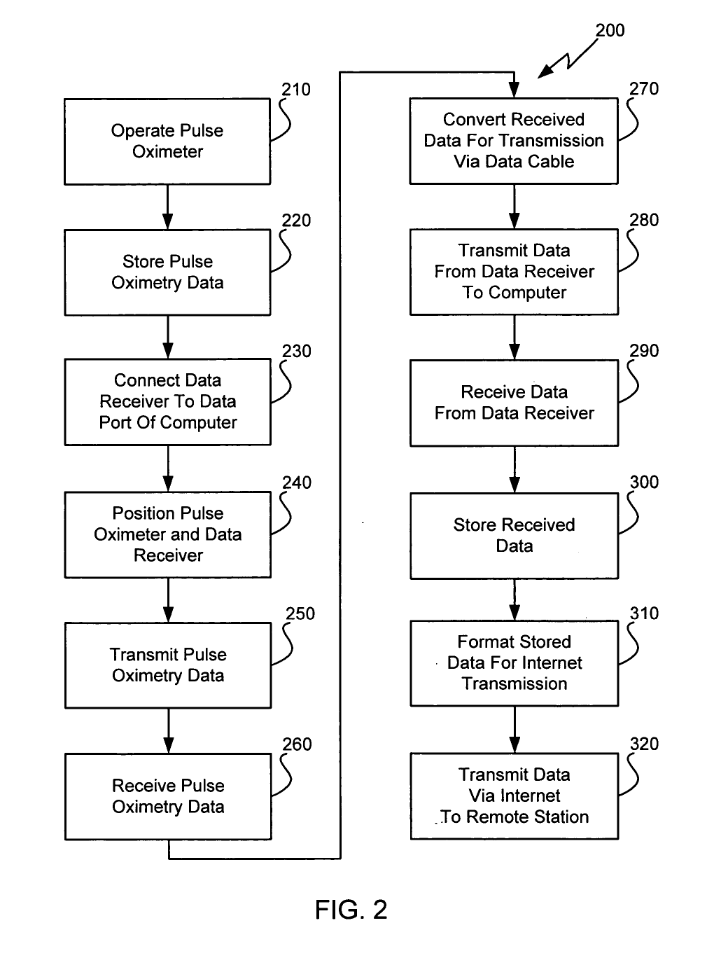

Referring now to FIG. 2 there is shown a flowchart illustrating one manner of using the wireless Internet connected pulse oximetry system 100 to provide pulse oximetry data 102 to a remote monitoring station via the Internet. The process (200) begins with operating (210) the pulse oximeter 110 to obtain pulse oximetry data 102 from a patient. In this regard, the pulse oximetry data 102 obtained may, for example, include digitized plethysmographic waveform data as well as identifying information indicating the date and time when the data was obtained and the identity of the patient from whom the data was obtained. In the illustrated embodiment, the obtained pulse oximetry data 102 is stored (220) in the memory 120 of the pulse oximeter 110. It should be noted that this step may not be necessary where the LED 114 and photodetector 132 are positioned in a suitable arrangement prior to obtaining the pulse oximetry data 102, in which case the pulse oximetry data 102 is transmitted from the pulse oximeter 110 to the optical data receiver 130 as it is obtained.

The optical data receiver 130 is connected (230) to the data port 144 of the computer 140 by the data cable 142, and the pulse oximeter 110 and optical data receiver 130 are positioned (240) in an appropriate relationship such that the LED 114 and photodetector 132 are respectively oriented with respect to one another (e.g., within the line of sight on one another) and within an appropriate distance apart from one another to permit reception by the photodetector 132 of optical signals transmitted from the LED 114.

Once the pulse oximeter 110 and optical data receiver 130 are appropriately positioned, the stored pulse oximetry data 102 is transmitted (250) from the LED 114. In this regard, the LED drive circuitry 116 may be operated to turn the LED 114 on and off to represent a series of digital values (e.g., 0 or 1) comprising the pulse oximetry data 102. The optical signal is received (260) by the photodetector 132 of the optical data receiver 130.

The optical data receiver 130 then converts (270) the optical signal received by the photodetector 132 into an appropriately formatted data signal for transmission via the data cable 142 to the data port 144 of the computer 140. In this regard, the conversion step (270) may involve converting the optical signal received by the photodetector 132 into an RS232 format serial data signal for transmission via the data cable 142 to a serial data port 144 of the computer 140. The converted data signal is then transmitted (280) from the optical data receiver 130 to the data port 144 of the computer 140 via the data cable 142. The conversion (270) and transmitting (280) steps may, for example, be performed simultaneously so that as optical data is received by the photodetector it is converted and transmitted to the data port 144 of the computer 140. In this regard, small portions (e.g. one or more bytes) of the optical data may be temporarily stored in a buffer memory of the optical data receiver prior to conversion and/or small portions (e.g., one or more bytes) of the converted data may be stored in the buffer memory prior to transmission to the data port 144 of the computer 140.

The pulse oximetry data 102 transmitted through the data cable 142 by the optical receiver 130 is received (290) by the data port 144 of the computer 140. The pulse oximetry data 102 received on the data port 144 is stored (300) by the computer in, for example, a data file saved on the data storage device 148 of the computer 140. In this regard, the data file may be named in a manner corresponding with patient identifying information and the date/time information included in the pulse oximetry data 102. Upon request by a remote monitoring station 150 via the Internet 104, the stored data is formatted (310) by the software module 146 into a format appropriate for transmission via the Internet to the remote monitoring station 150. In this regard, the stored data may, for example, be formatted in accordance with protocols such as the hypertext transfer protocol (HTTP) or the file transfer protocol (FTP). The formatted data is then transmitted (320) by the computer 140 via the Internet 104 to the requesting remote monitoring station 150. It should be noted that the formatting step (310) may alternatively be performed before receiving a request for the stored pulse oximetry data 102 and the pulse oximetry data 102 may be stored in the Internet transmittable form. Also, the step of storing (300) the pulse oximetry data 102 may be omitted. In this regard, the pulse oximetry data 102 may be formatted (310) for transmission and transmitted (320) via the Internet 104 to a remote monitoring station 150 as it is received on the data port 144 from the optical data receiver 130.

Although the steps of the process (200) are shown in FIG. 2 in a particular order, it should be noted that the steps need not necessarily be performed in the order described. For example, the steps of connecting (230) the optical data receiver 130 to the data port 144 of the computer 140 and positioning (240) the pulse oximeter 110 and optical data receiver 130 in an appropriate relationship may be performed in the order described, simultaneously, or in the opposite order. Likewise, one or both of the connecting (230) and positioning (240) steps may be performed before or after the steps of operating (210) the pulse oximeter 110 and storing (220) the pulse oximetry data 102. Further, as previously mentioned, the steps of storing the pulse oximetry data (220, 300) in the pulse oximeter 110 and/or on the data storage device 148 of the computer 140 may be omitted.

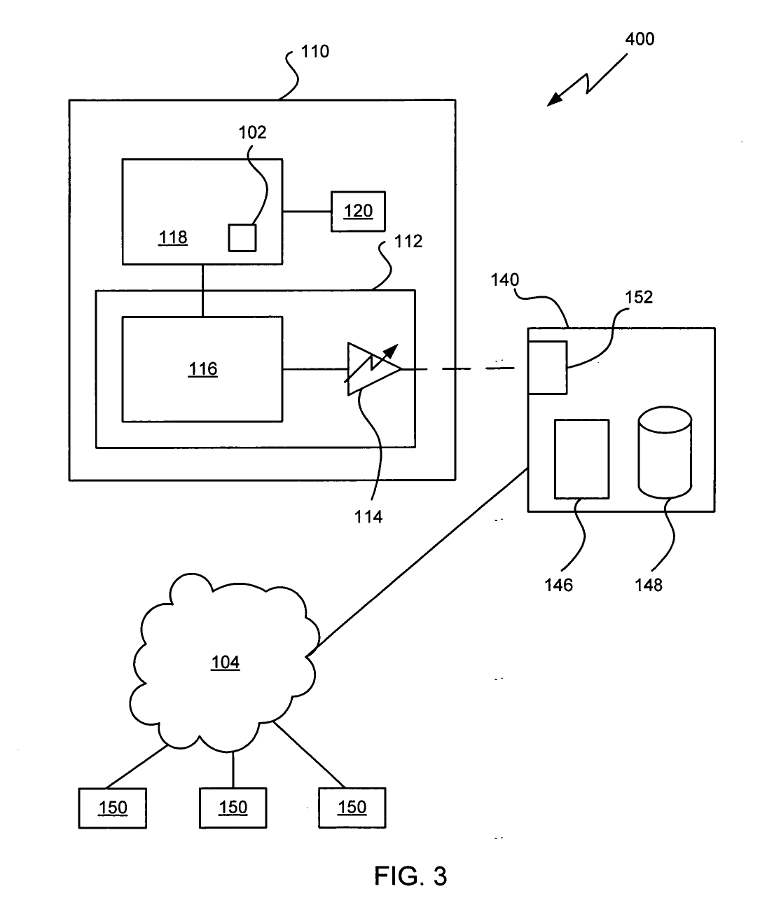

Referring now to FIG. 3 there is shown a block diagram of an embodiment of a wireless Internet connected pulse oximetry system 400 that does not include an optical data receiver 130. In this regard, system 400 generally includes a pulse oximeter 110 and a computer 140 (e.g., a desktop, laptop or handheld computer or the like). The pulse oximeter 110 includes an optical data transmitter 112 which may, for example, comprise an infra-red light emitting diode (LED) 114 and related LED drive circuitry 116. The LED drive circuitry 116 is operable to receive pulse oximetry data 102 (e.g., a digitized plethysmographic waveform) from a processor 118 of the pulse oximeter 110 and modulate the LED 114 to optically transmit the oximetry data 102. In this regard, the pulse oximeter 110 may comprise a relatively small, portable pulse oximeter unit having a built in LED 114 and LED drive circuitry 116 such as the Datex-Ohmeda TUFFSAT® handheld pulse oximeter.

The computer 140 includes an optical data port 152 (e.g., an IR port) for receiving the optically transmitted pulse oximetry data 102 directly from the LED 114 of the pulse oximeter 110. In this regard, LED 114 and optical data port 152 should generally be maintained in a line of sight relationship with each other and within a suitable range of one another in order for the optically transmitted pulse oximetry data 102 to be received. In this regard, the pulse oximeter 110 may also include a data storage device 120 (e.g., random access memory, flash memory) for storing the pulse oximetry data 102 for some period of time until the LED 114 of the pulse oximeter 110 and the optical data port 152 of the computer 140 can be brought into a suitable relationship with one another at which time the stored pulse oximetry data 102 may be transmitted.

The computer 140 is connected to the Internet 104 via, for example, a modem connected to an Internet Service Provider server or a server of a local area network connected to the Internet 104. The computer 140 includes an appropriately configured software module 146 that, when executed by the computer 140, takes the pulse oximetry data 102 received on the optical data port 152 and formats the pulse oximetry data 102 for transmission over the computer's Internet connection to other devices (e.g., remote pulse oximetry monitoring and/or processing devices) connected to the Internet 104. Thus, pulse oximetry data 102 obtained by the pulse oximeter 110 is made available via the Internet 104 to remote monitoring stations 150 (e.g., other computers) that are geographically remote from the location of the patient being monitored by the pulse oximeter 110. In this regard, the computer 140 may include a data storage device 148 (e.g., a hard drive or a CDRW drive) for storing the pulse oximetry data 102 for some period of time until the pulse oximetry data 102 is requested by a remote monitoring station 150, at which time the pulse oximetry data 102 is transmitted by the computer 140 via the Internet 104 to the remote monitoring station 150.

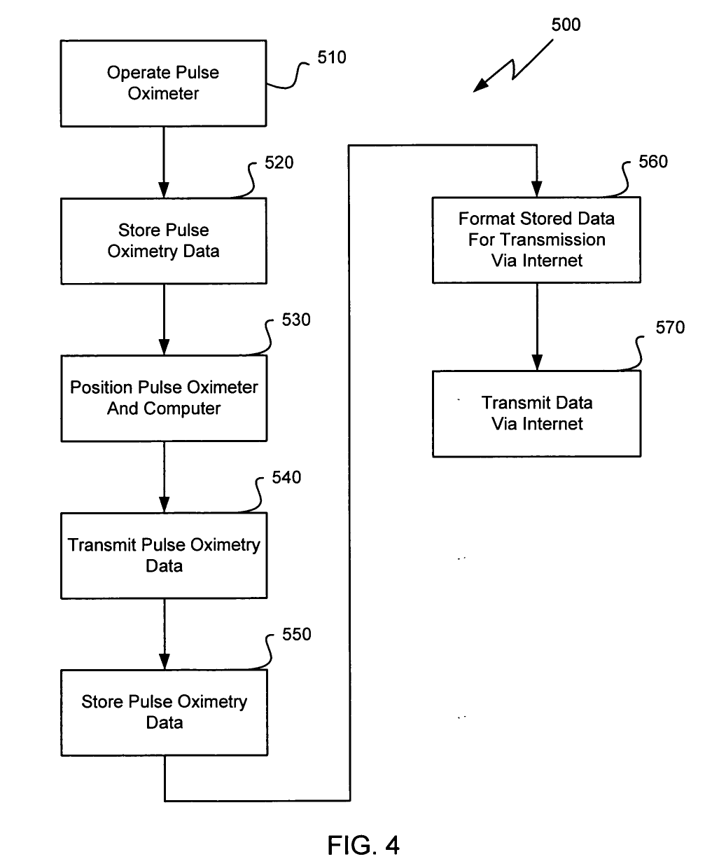

Referring now to FIG. 4 there is shown a flowchart illustrating one manner of using the wireless Internet connected pulse oximetry system 400 to provide pulse oximetry data 102 to a remote monitoring station 150 via the Internet. The process (500) begins with operating (510) the pulse oximeter 110 to obtain pulse oximetry data 102 from a patient. In this regard, the pulse oximetry data 102 obtained may, for example, include digitized plethysmographic waveform data as well as identifying information indicating the date and time when the data was obtained and the identity of the patient from whom the data was obtained. In the illustrated embodiment, the obtained pulse oximetry data 102 is stored (520) in the memory 120 of the pulse oximeter 110. It should be noted that this step may not be necessary where the LED 114 and optical data port 152 of the computer 140 are positioned in a suitable arrangement prior to obtaining the pulse oximetry data 102, in which case the pulse oximetry data 102 is transmitted from the pulse oximeter 110 to the optical data port 152 as it is obtained.

The pulse oximeter 110 and computer 140 are positioned (530) in an appropriate relationship such that the LED 114 and optical data port 152 are respectively oriented with respect to one another (e.g., within the line of sight on one another) and within an appropriate distance apart from one another to permit reception by the optical data port 152 of optical signals transmitted from the LED 114. Once the pulse oximeter 110 and computer 140 are appropriately positioned, the stored pulse oximetry data 102 is transmitted (540) from the LED 114. In this regard, the LED drive circuitry 116 may be operated to turn the LED 114 on and off to represent a series of digital values (e.g., 0 or 1) comprising the pulse oximetry data 102. The optical signal is received (550) by the optical data port 152 of the computer 140.

The pulse oximetry data 102 received on the optical data port 152 is stored (550) by the computer 140 in, for example, a data file saved on the data storage device 148 of the computer 140. In this regard, the data file may be named in a manner corresponding with patient identifying information and the date/time information included in the pulse oximetry data 102. Upon request by a remote monitoring station 150 via the Internet 104, the stored data is formatted (560) by the software module 146 into a format appropriate for transmission via the Internet to the remote monitoring station 150. In this regard, the stored data may, for example, be formatted in accordance with protocols such as the hypertext transfer protocol (HTTP) or the file transfer protocol (FTP). The formatted data is then transmitted (570) by the computer 140 via the Internet 104 to the requesting remote monitoring station. It should be noted that the formatting step (560) may alternatively be performed before receiving a request for the stored pulse oximetry data 102 and the pulse oximetry data 102 may be stored in the Internet transmittable form. Also, the step of storing (550) the pulse oximetry data 102 may be omitted. In this regard, the pulse oximetry data 102 may be formatted (560) for transmission and transmitted (570) via the Internet 104 to a remote monitoring station 150 as it is received on the optical data port 152 from the pulse oximeter 110.

Although the steps of the process (500) are shown in FIG. 4 in a particular order, it should be noted that the steps need not necessarily be performed in the order described. For example, the step of positioning (530) the pulse oximeter 110 and the computer 140 in an appropriate relationship may be performed before operating (510) the pulse oximeter 110. Further, as previously mentioned, the steps of storing the pulse oximetry data (520, 550) in the pulse oximeter 110 and/or on the data storage device 148 of the computer 140 may be omitted.

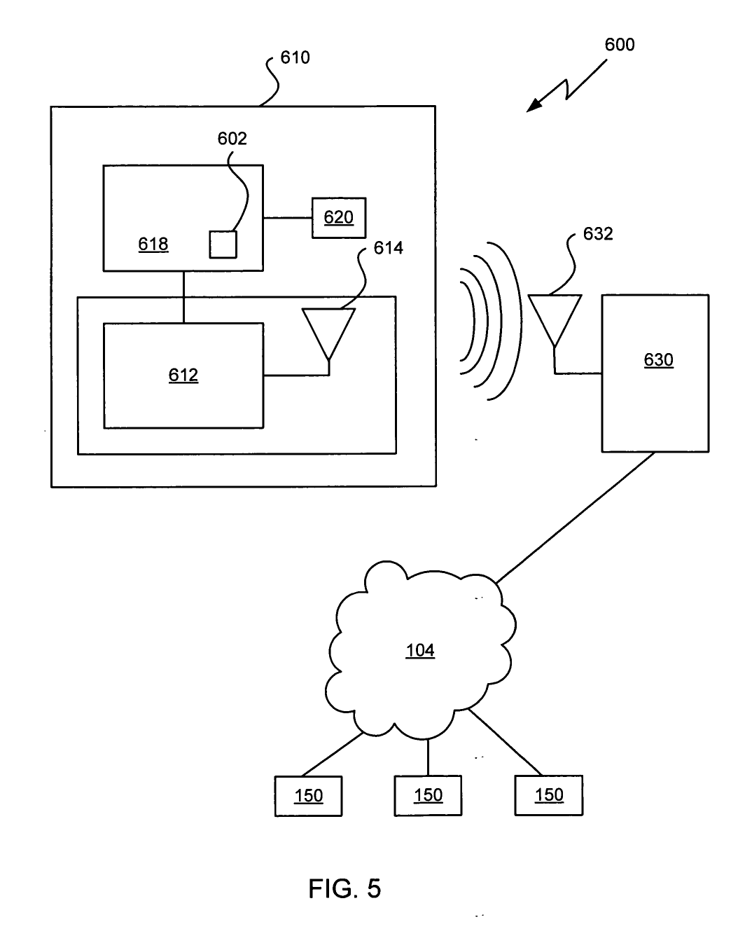

Referring now to FIG. 5 there is shown a block diagram of an embodiment of a wireless Internet connected pulse oximetry system 600 employing wireless RF technology (e.g. WiFi technology). In this regard, system 600 generally includes a pulse oximeter 610 and a wireless network RF receiver 630. The pulse oximeter 610 includes an RF transmitter 612 coupled to an antenna 614. The RF transmitter 612 is operable to receive pulse oximetry data 602 (e.g., a digitized plethysmographic waveform) from a processor 618 of the pulse oximeter 610 and modulate an RF carrier signal to transmit the oximetry data 602.

The RF receiver 630 includes an antenna 632 for receiving the RF transmitted pulse oximetry data 602 signal broadcast by the RF transmitter 612 of the pulse oximeter 610. In order to achieve accurate transmission of the pulse oximetry data 602, the pulse oximeter 610 and RF receiver 630 should generally be within suitable RF broadcast range with each other. In this regard, the pulse oximeter 610 may also include a data storage device 620 (e.g., random access memory, flash memory) for storing the pulse oximetry data 602 for some period of time until the pulse oximeter 610 and RF receiver 630 can be brought into a suitable range with one another at which time the stored pulse oximetry data 602 may be transmitted.

The RF receiver 630 is connected to the Internet 104 via, for example, a modem connected to an Internet Service Provider server or a server of a local area network connected to the Internet 104. The RF receiver 630 receives the pulse oximetry data 602 and formats the pulse oximetry data 602 for transmission to other devices (e.g., remote pulse oximetry monitoring and/or processing devices) connected to the Internet 104. Thus, pulse oximetry data 602 obtained by the pulse oximeter 610 is made available via the Internet 104 to remote monitoring stations 150 (e.g., other computers) that are geographically remote from the location of the patient being monitored by the pulse oximeter 110.

While various embodiments of the present invention have been described in detail, further modifications and adaptations of the invention may occur to those skilled in the art. However, it is to be expressly understood that such modifications and adaptations are within the spirit and scope of the present invention.

What is claimed is:

1. A wireless network connected pulse oximetry system comprising:
a pulse oximeter including an optical data transmitter, said pulse oximeter being operable to obtain pulse oximetry data from a patient, said optical data transmitter being operable to optically transmit the pulse oximetry data;
a computer interconnectable with a global data network, said computer including a data port;
an optical data receiver, said optical data receiver being connectable with said data port of said computer via a data cable, said optical data receiver being operable to receive optically transmitted pulse oximetry data from said pulse oximeter and convert the received pulse oximetry data for transmission via the data cable to said data port of said computer; and
a software module executable by said computer, said software module enabling said computer to format the pulse oximetry data received on said data port for transmission via the global data network to a remote monitoring station.

2. The system of claim 1 wherein said optical data transmitter comprises an infra-red LED and LED drive circuitry operable to modulate said LED.

3. The system of claim 1 wherein said optical data receiver includes a photodetector sensitive to infra-red wavelength optical signals.

4. The system of claim 1 further comprising:
a data storage device for storing the pulse oximetry data after the pulse oximetry data is obtained from the patient.

5. The system of claim 4 wherein said data storage device comprises a memory chip included in said pulse oximeter.

6. The system of claim 1 further comprising:
a data storage device for storing the pulse oximetry data after the pulse oximetry data is received on said data port.

7. The system of claim 6 wherein said data storage device comprises a hard drive, a floppy drive, an optical media drive, or a tape drive included in said computer.

8. The system of claim 1 wherein the global data network comprises the Internet.

9. A method of providing pulse oximetry data obtained by a pulse oximeter to a monitoring station that is remote from a patient monitored by the pulse oximeter, said method comprising the steps of:
connecting an optical data receiver by a data cable to a data port of a computer interconnectable with a global data network;
positioning the pulse oximeter and the optical data receiver for optical transmission of the pulse oximetry data therebetween;
optically transmitting the pulse oximetry data from the pulse oximeter;
receiving the optically transmitted pulse oximetry data with the optical data receiver;
converting the received optically transmitted pulse oximetry data to a format appropriate for transmission via the data cable to the data port of the computer;
transmitting the converted pulse oximetry data from the optical data receiver to the data port of the computer;
receiving the converted pulse oximetry data on the data port of the computer;
formatting the pulse oximetry data received on the data port for transmission over the global data network; and
transmitting the formatted pulse oximetry data over the global data network to the remote monitoring station.

10. The method of claim 9 wherein said step of positioning includes the step of aligning an LED of the pulse oximeter in a line of sight relationship with a photodetector of the optical data receiver.

11. The method of claim 9 further comprising the step of:
operating the pulse oximeter to obtain the pulse oximetry data.

12. The method of claim 11 wherein said step of optically transmitting the pulse oximetry data is performed simultaneously with said step of operating the pulse oximeter to obtain the pulse oximetry data.

13. The method of claim 11 further comprising the step of:
storing the pulse oximetry data obtained in said operating step on a data storage device of the pulse oximeter.

14. The method of claim 13 wherein said step of optically transmitting the pulse oximetry data is performed after said step of storing the pulse oximetry data.

15. The method of claim 9 wherein said steps of receiving the converted pulse oximetry data on the data port of the computer, formatting the pulse oximetry data received on the data port for transmission over the global data network, and transmitting the formatted pulse oximetry data over the global data network to the remote monitoring station are performed simultaneously.

16. The method of claim 9 further comprising the step of:
storing the pulse oximetry data on a data storage device of the computer.

17. The method of claim 16 wherein said step of formatting the pulse oximetry data is performed before said step of storing the pulse oximetry data on a data storage device of the computer.

18. The method of claim 16 wherein said step of formatting the pulse oximetry data is performed after said step of storing the pulse oximetry data on a data storage device of the computer.

19. The method of claim 16 wherein said step of transmitting the formatted pulse oximetry data over the global data network to the remote monitoring station is performed after said step of storing the pulse oximetry data on a data storage device of computer.

20. The method of claim 9 wherein in said step of transmitting the formatted pulse oximetry data over the global data network, the global data network comprises the Internet.

21. A wireless network connected pulse oximetry system comprising:
a pulse oximeter including an optical data transmitter, said pulse oximeter being operable to obtain pulse oximetry data from a patient, said optical data transmitter being operable to optically transmit the pulse oximetry data;
a computer interconnectable with a global data network, said computer including an optical data port operable to receive optically transmitted data; and
a software module executable by said computer, said software module enabling said computer to format the pulse oximetry data received on said optical data port for transmission via the global data network to a remote monitoring station.

22. The system of claim 21 wherein said optical data transmitter comprises an infra-red LED and LED drive circuitry operable to modulate said LED.

23. The system of claim 21 wherein said optical data port of said computer includes a photodetector sensitive to infra-red wavelength optical signals.

24. The system of claim 21 further comprising:
a data storage device for storing the pulse oximetry data after the pulse oximetry data is obtained from the patient.

25. The system of claim 24 wherein said data storage device comprises a memory chip included in said pulse oximeter.

26. The system of claim 21 further comprising:
a data storage device for storing the pulse oximetry data after the pulse oximetry data is received on said optical data port.

27. The system of claim 26 wherein said data storage device comprises a hard drive, a floppy drive, an optical media drive, or a tape drive included in said computer.

28. The system of claim 21 wherein the global data network comprises the Internet.

29. A method of providing pulse oximetry data obtained by a pulse oximeter having an optical data transmitter to a monitoring station that is remote from a patient monitored by the pulse oximeter, said method comprising the steps of:
positioning the pulse oximeter and a computer having an optical data port and interconnectable with a global data network for optical transmission of the pulse oximetry data therebetween;
optically transmitting the pulse oximetry data from the pulse oximeter;
receiving the optically transmitted pulse oximetry data on the optical data port of the computer;
formatting the pulse oximetry data received on the optical data port for transmission over the global data network; and
transmitting the formatted pulse oximetry data over the global data network to the remote monitoring station.

30. The method of claim 29 wherein said step of positioning includes the step of aligning an LED of the pulse oximeter in a line of sight relationship with a photodetector of the optical data port of the computer.

31. The method of claim 29 further comprising the step of:
operating the pulse oximeter to obtain the pulse oximetry data.

32. The method of claim 31 wherein said step of optically transmitting the pulse oximetry data is performed simultaneously with said step of operating the pulse oximeter to obtain the pulse oximetry data.

33. The method of claim 31 further comprising the step of:
storing the pulse oximetry data obtained in said operating step on a data storage device of the pulse oximeter.

34. The method of claim 33 wherein said step of optically transmitting the pulse oximetry data is performed after said step of storing the pulse oximetry data.

35. The method of claim 29 wherein said steps of receiving the optically transmitted pulse oximetry data on the optical data port of the computer, formatting the pulse oximetry data received on the optical data port for transmission over the global data network, and transmitting the formatted pulse oximetry data over the global data network to the remote monitoring station are performed simultaneously.

36. The method of claim 29 further comprising the step of:
storing the pulse oximetry data on a data storage device of the computer.

37. The method of claim 36 wherein said step of formatting the pulse oximetry data is performed before the step of storing the pulse oximetry data on a data storage device of the computer.

38. The method of claim 36 wherein said step of formatting the pulse oximetry data is performed after the step of storing the pulse oximetry data on a data storage device of the computer.

39. The method of claim 36 wherein said step of transmitting the formatted pulse oximetry data over the global data network to the remote monitoring station is performed after the step of storing the pulse oximetry data on a data storage device of the computer.

40. The method of claim 29 wherein in said step of transmitting the formatted pulse oximetry data over the global data network, the global data network comprises the Internet.

41. A wireless network connected pulse oximetry system comprising:
a pulse oximeter including an RF data transmitter, said pulse oximeter being operable to obtain pulse oximetry data from a patient, said RF data transmitter being operable to broadcast an RF signal modulated to include the pulse oximetry data; and
an RF data receiver interconnectable with a global data network, said RF data receiver being operable to receive the RF signal broadcast by said pulse oximeter and to convert the pulse oximetry data from the received RF signal for transmission via the global data network to a remote monitoring station.

42. The system of claim 41 wherein said RF data transmitter and said RF receiver comprise WiFi network devices.

43. The system of claim 41 further comprising:
a data storage device for storing the pulse oximetry data in the pulse oximeter after the pulse oximetry data is obtained from the patient.

44. The system of claim 43 wherein said data storage device comprises a memory chip included in said pulse oximeter.

45. The system of claim 41 wherein the global data network comprises the Internet.
